(19) United States
(12) Patent Application Publication
(10) Pub. No.: US 2004/0028734 A1
(43) Pub. Date: Feb. 12, 2004

(54) Use of anti-muscarinic agents and calcium channel blockers in combination

(76) Inventors: Robin Mark Bannister, Essex (GB); Nicola Cooper, Essex (GB)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/415,427

(22) PCT Filed: Dec. 7, 2001
(86) PCT No.: PCT/GB01/05450

(30) Foreign Application Priority Data
Dec. 7, 2000 (GB) 0029903.2

(51) Int. Cl. A61K 31/554; A61K 31/4745; A61K 31/455; A61K 31/401; A61K 31/277

(52) U.S. Cl. 424/468; 514/211.07; 514/291; 514/355; 514/423; 514/519

(57) ABSTRACT

An anti-muscarinic agent and a calcium channel blocker are suitable for simultaneous, sequential or separate use in the treatment of a muscle tone disorder or a proliferative, inflammatory or secretory condition.

### FIELD OF THE INVENTION

This invention relates to the use of anti-muscarinic agents and calcium channel blockers in combination.

### BACKGROUND OF THE INVENTION

Atropine is a very well established anti-muscarinic agent. The drug exhibits all the pharmacological and toxicological effects of this class of therapeutic agents. The drug itself is a potent bronchodilator, acting on the peripheral smooth muscle lining of the bronchi. In addition, the drug is known as an antisialogogue, being used to dry mucous secretions, as well as having central effects as exemplified by its use in motion sickness, and in schizophrenia. The ubiquitous nature of the muscarinic receptors and the indiscriminate nature of the binding of atropine to these receptors are also responsible for its side-effects. Side-effects include constipation, urinary retention, confusion, blurred vision and dry mouth.

Muscarinic antagonism is utilised in the treatment of respiratory disease. A number of successful therapies are based upon the bronchodilatory and antisecretory properties of anti-muscarinic agents.

Direct evidence of anti-proliferative activity by muscarinic antagonism comes from the demonstration by Qiu et al (Sheng Li Hsueh Pao 1995, 275-80) that T lymphocytes undergo enhanced proliferation through the mediation by acetylcholine, and that atropine abolishes this effect completely.

The clinical utility of anti-muscarinic therapy remains limited, due to systemic side-effects. These can be managed by local administration to diseased tissue. Such a mode of administration occurs with the bronchodilator ipratropium when used in inhaled form. Alternatively, modifications to receptor selectivity can be considered, for example the use of selective M3 antagonists in asthma. However, both of these approaches have major drawbacks either in terms of poor tissue accessability or persistent side-effects.

WO-A-98/00119 discloses the use of agents that affect non-neuronal acetylcholine functions, for the treatment of skin ailm nts. It also discloses that topically effective antagonists of muscarinic receptors, including ipratropium, are useful for the treatment of skin ailments. Various skin ailments that are disclosed include atopic dermatitis, neurodermatitis, psoriasis and cholinergic urticaria.

WO-A-01/10427 discloses that skin conditions are treated by the topical application of a quaternary ammonium or other compound having anti-muscarinic activity, a high dipole moment (greater than 4D) and high anti-proliferation activity (at least 50% inhibition at 10 μM). It may also have high receptor-binding activity (half-life for receptor dissociation greater than 0.11 h at M1).

WO-A-01/76575 discloses a pharmaceutical composition for pulmonary administration, comprising an anti-muscarinic agent. The preferred agent is glycopyrrolate. The compositon is particularly suitable for the treatment of asthma, cystic fibrosis and chronic obstructive pulmonary disease.

Calcium channel blocking has for some time been an attractive approach to the treatment of respiratory inflammatory diseases. However, the available agents have lacked the potency for effective treatment and no effective therapies have been developed.

### SUMMARY OF THE INVENTION

It has now been appreciated that there is an alternative and attractive method of overcoming the problems described above. According to this invention, calcium channel blockade is used in conjunction with muscarinic antagonists, e.g. those that have long receptor-binding characteristics, to provide both longer duration of action with enhanced effect and the use of lower doses. When administered topically, the local effect of the drug can be significantly enhanced whilst ensuring that systemic and side-effects can be further minimised.

The invention is based on the discovery that the action of calcium channel blocking agents can be potentiated by the combined activity of muscarinic antagonism, and vice versa. Using the guinea pig functional model of smooth muscle relaxation, it has been demonstrated that a small addition of an anti-muscarinic agent causes almost a ten-fold increase in effect of the calcium channel blocking agent. This for the first time allows the consideration of calcium channel blocking agents as components of effective treatments for respiratory disease.

The respective active agents used in the present invention may be formulated together, e.g. in “kit” form. Alternatively, one may be administered to a subject who is undergoing therapy with the other.

Using these agents in combination allows a precise tailoring of activity for most desirable patient outcome. For example, for patients with additional complications in which anti-muscarinic activity gives rise to additional side-effects, a threshold level of anti-muscarinic agent can be used. Such patient groups include those suffering from cardiac complaints, glaucoma and problems with gut motility. In addition, patients suffering from cholinergic load as a result of concomitant medicine will also benefit from bronchial smooth muscle relaxation as a result of minimal but threshold muscarinic antagonism and maximal calcium channel blockade. Conversely, patient groups with severely compromised respiratory function benefit from maximal muscarinic antagonism. The currently available agent (ipratropium) provides only short term maximal antagonism. However, the calcium channel blocking agents are known to be metabolically stable in the lung and in some cases such as verapamil are sequestered and accumulate in lung tissue. The addition of an appropriate calcium channel blocking agent provides substantial background smooth muscle relaxation and less reliance upon precise dosing intervals for best patient benefit.

The use of certain anti-muscarinic components leads to a low plasma half-life, thus reducing their side-effects. The route of administration of these molecules can be used to further limit systemic exposure and side-effects.

### DESCRIPTION OF PREFERRED EMBODIMENTS

The present invention is particularly suitable to topical administration, e.g. dermally, to the lung, and to the gastrointestinal tract. For dermal use, and as described in WO-A-01/10427, keratinocyte proliferation may be inhibited, using anti-muscarinic agents. Preferred such agents have a dipole moment of greater than 4.0, since they show limited systemic exposure due to poor passage across the skin to the circulatory system.

Anti-muscarinic agents for use in this invention preferably also have high receptor-binding affinity. A long duration of action is extremely desirable for a topically applied drug to treat local conditions. This leads to low reapplication rates of medication, which in turn ensures minimum disturbance to patient lifestyle, and high patient compliance. Compounds with high receptor binding affinity include glycopyrrolate, ipratropium and tiotropium.

At the clinical level, glycopyrrolate is known to have a longer duration of action in muscarinic antagonism than ipratropium; see J. Allergy Clin. Immunol. (1988)82:115. In addition, in Frey's syndrome, a two day duration of action from a single dermal application appears to be common, in the use of glycopyrrolate.

Disse et al, Life Sciences (1993) 52/5-6:537-544, compared the dissociation rates of ipratropium and tiotropium. For muscarinic receptor M1, the half-lives were 0.11 h and 14.6 h; for M3, they were 0.26 h and 34.7 h, respectively. The relatively low off-rate and long half-life for tiotropium are responsible for its very long duration of action in smooth muscle relaxation involving muscarinic antagonism.

More particularly, suitable anti-muscarinic agents for dermal use at least in the invention may initially be identified by the Assay Protocol described in WO-A-01/10427, which is a model of psoriasis and thus of a proliferative skin condition. An agent for use in the invention preferably has an IC₅₀ value below 100 μM, most preferably below 10 μM, e.g. below 1 μM, and most preferably below 100 nM.

Anti-muscarinic agents that are suitable for administration to the lung are described in WO-A-01/76575. Examples of anti-muscarinic agents that can be used in the invention include ambutonium, benzilonium, dibutoline, diphemanil, emepronium, glycopyrrolate, isopropamide, lachesine, mepenzolate, methantheline, oxyphenonium, oxytroprium, penthienate, phenthimentonium, pipenzolate, poldine, tiemonium, tiotropium, tricyclamol and tridihexethyl. Glycopyrrolate is preferred.

The calcium channel blockade may be provided by any of a number of agents that are known to those skilled in the art. Examples include diltiazem, verapamil and dihydropyridine drugs. Other such agents will typically have at least substantially the same (at least 50%) of the activity of the given agents, in an assay for calcium channel blocking activity.

These and other compounds for use in the invention may be provided in the form of a free base or salt. All such forms are within the scope of the invention, and in particular salts, organic and inorganic, are included. For example, quaternary ammonium compounds may be provided as a halide or other salt.

Many compounds suitable for use in the invention exhibit isomerism, whether optical or structural (stereoisomerism/regioisomerism). Such compounds include glycopyrrolate and tiotropium. Application of a single isomer or a non-stoichiometric mixture of isomers, e.g. a non-racemic mixture, in the case of optical isomers, may optimise the desired antiproliferative activity.

This invention is of particular value in the treatment of respiratory conditions, including respiratory inflammation and respiratory proliferation. In particular, the invention may be used in the treatment of chronic obstructive pulmonary disease (COPD), asthma or cystic fibrosis, or associated conditions. For the treatment of each condition, the active agents may be administered by inhalation.

The active agent may also be administered by the oral route. This can be used in therapy where the condition to be treated is a muscle tone disorder, e.g. of the gastrointestinal or urinary tract, or which involves gut motility or urinary incontinence. Here and in general, the anti-secretory and smooth muscle action of the new combination can be utilised.

Dermal conditions that may be treated include all forms of psoriasis, including psoriatic and scalp arthritis, skin cancer, melanoma, pemphigus, atopic dermatitis, neuro-dermatitis, eczema, contact dermatitis, acne, leprosy, seborrheic dermatitis, lupus and urticaria. The invention is particularly suited to the treatment of topical proliferative conditions such as psoriasis. Treatment may be combined with radiological therapy. Alternatively or in addition, treatment may be combined with a conventional agent, of which examples include steroids, vitamins A and D and their analogues, salicylates, anthralines and coal tar preparations.

Conventional topical formulations and administration techniques may be used. For example, for dermal use, suitable compositions include, but are not limited to, creams, ointments, gels, shampoos, lotions, ionotophoresis, patches and emollients. The two active agents may be formulated in a mixture of independently, e.g. in a kit. In particular, this invention provides anti-muscarinic agents to treat skin condition by topical administration, in which the drug is placed in a formulation system in which the drug flux across the skin is maintained at such a rate that systemic blood levels are retained at a low level. However, the drug flux is maintained at a level to effect topical activity in the skin. In this way, anti-muscarinic agents may be used that would otherwise be limited by their side-effects.

Devices and formulations suitable for delivery by inhalation are known to the skilled person. The composition may be prepared for delivery as an aerosol in a liquid propellant, for example for use in a pressurised metered dose inhaler (PMDI's). Propellants suitable for use in a PMDI are known to the skilled person, and include CFC-12, HFA-134a, HFA-227, HCFC-22 (difluorochloromethane), HFA-152 (difluoroethane and isobutane).

In a preferred embodiment of the invention, the compositions are in a dry powder form, for delivery using a dry powder inhaler (DPI). Dry powder inhalers are known. The dry powders for use in the inhalers will usually have a mass medium aerodynamic diameter of less than 30 μm, preferably less than 20 μm and more preferably less than 10 μm. Microparticles having aerodynamic diameters in the range of 5 to 0.5 μm will generally be deposited in the respiratory bronchioles, whereas smaller particles having aerodynamic diameters in the range of 2 to 0.05 μm are likely to be deposited in the alveoli.

The microparticles may also be formulated with additional excipients to aid delivery and release. For example, in the context of dry powder formulations, the microparticles may be formulated with additional large carrier particles which aid the flow from the dry powder inhaler into the lung. Large carrier particles are known, and include lactose particles having a mass medium aerodynamic diameter of greater than 90 μm. Alternatively, the hydrophobic microparticles may be dispersed within a carrier material. For example, the hydrophobic microparticles may be dispersed within a polysaccharide matrix, with the overall composition formulated as microparticles for direct delivery to the lung. The polysaccharide acts as a further barrier to the immediate release of the glycopyrrolate component. This may further aid the controlled release process. Suitable carrier materials will be apparent to the skilled person and include any pharmaceutically acceptable insoluble or soluble material, including polysaccharides. An example of a suitable polysaccharide is xantham gum.

The amount of the active agent to be used will depend on the usual factors, such as the potency of the agent, the nature and state of the condition to be treated, the state of the patient, etc. All these factors can be taken into account, and the relevant dose determined accordingly, by the skilled man.

The following study provides evidence on which this invention is based.

Study

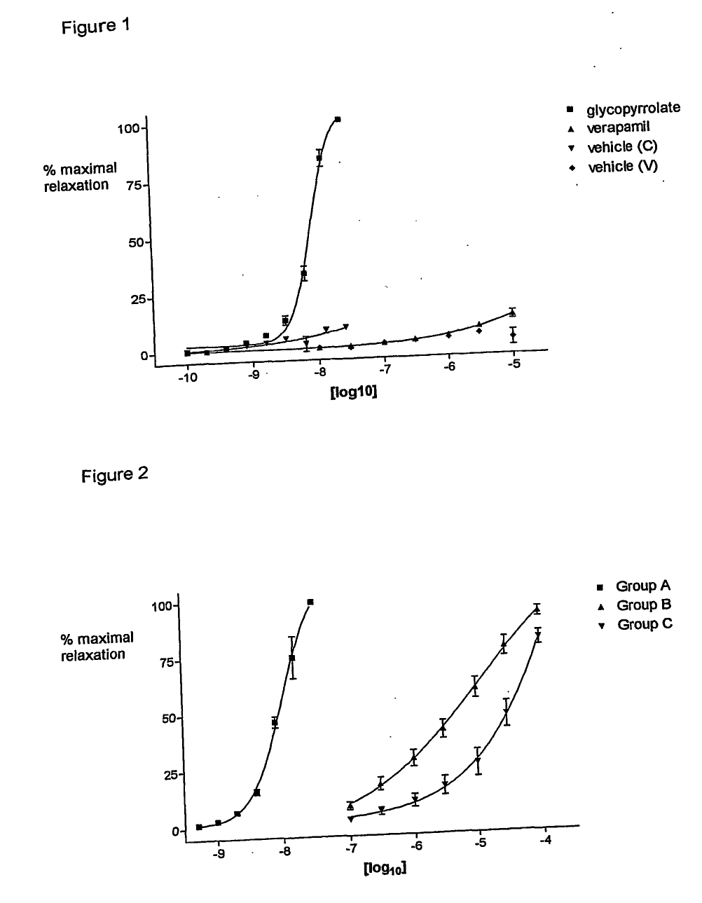

Guinea pig tracheal strip preparations were pre-contracted with 5×10⁻⁶ M carbachol for 10 minutes, to induce tone, before incubation with drug. The relaxation (% maximal of carbachol-induced tone) induced by the treatment is shown in FIG. 1; this drawing shows cumulative concentration-relaxation curves generated using a 5 minute dose-cycle. Relaxation is expressed as % maximal relaxation induced by 5×10⁻⁴ M papaverine after completion of concentration-response curves. Maximal relaxation of time-matched control tissues is 3.5±1.9% after 50 minutes (35 minutes of dose-cycle). EC₅₀ values:

| | -Log EC₅₀ | s.e.m | EC₅₀ (M) | 95% C.I. |
|---|---|---|---|---|
| glyco- | 8.04 | 0.03 | 9.23 × 10⁻⁹ | 7.9 × 10⁻⁹ − 1.1 × 10⁻⁸ |
| pyrrolate | | | | |
| verapamil | 4.56 | 0.35 | 2.73 × 10⁻⁵ | 1.8 × 10⁻⁵ − 4.0 × 10⁻⁵ |

Further experiments were conducted, to investigate the effect of pre-treatment with threshold concentrations on concentration-response curves to glycopyrrolate and verapamil. Again, guinea pig tracheal strip preparations were pre-contracted with 5×10⁻⁶ M carbachol for 10 minutes before incubation with the pre-treatment drug. The relaxation (% maximal of carbachol-induced tone) induced by the pre-treatment is shown in FIG. 2, for the following groups.

| Group | Pre-treatment | EC₅₀ (M) | % Relaxation | DRC to |
|---|---|---|---|---|
| A | verapamil | 5 × 10⁻⁶ M | 6.6 ± 0.5 | glycopyrrolate |
| B | glycopyrrolate | 4 × 10⁻⁹ M | 6.5 ± 0.8 | verapamil |
| C | glycopyrrolate | 4 × 10⁻¹⁰ M | −0.7 ± 1.4 | verapamil |

In summary, the calcium antagonist verapamil is not a potent inhibitor of carbachol-induced bronchoconstriction in the guinea pig tracheal strip (EC₅₀ 2.73×10⁻⁵). However, addition of a threshold dose of glycopyrrolate (a potent muscarinic antagonist) causes an almost 10-fold shift in EC₅₀ for the drug (EC₅₀ 5×10⁻⁶).

These results demonstrate the utility of calcium antagonists in combination with muscarinic antagonists in human respiratory disease where a bronchodilator may be necessary, e.g. asthma and COPD. It is also more general evidence of this invention.

1. A product comprising an anti-muscarinic agent and a calcium channel blocker, for simultaneous, sequential or separate use in the treatment of a muscle tone disorder or a proliferative, inflammatory or secretory condition.

2. A product in the form of a medicament comprising an anti-muscarinic agent and a calcium channel blocker.

3. A product according to claim 1 or claim 2, in a form suitable for topical administration.

4. A product according to claim 1 or claim 2, in a form suitable for inhaled or systemic administration.

5. A product according to any preceding claim, wherein the anti-muscarinic agent and the calcium channel blocker are formulated to have different release rates.

6. A product according to any preceding claim, wherein the anti-muscarinic agent is glycopyrrolate.

7. A product according to any of claims 1 to 5, wherein the anti-muscarinic agent is tiotropium.

8. A product according to any of claims 1 to 7, wherein the calcium channel blocker is diltiazem.

9. A product according to any of claims 1 to 7, wherein the calcium channel blocker is verapamil.

10. A product according to any of claims 1 to 7, wherein the calcium channel blocker is a dihydropyridine drug.

11. A product according to any preceding claim, for the treatment of a proliferative, inflammatory or secretory condition.

12. Use of an anti-muscarinic agent for the manufacture of a medicament for use in the treatment of a muscle tone disorder or a proliferative, inflammatory or secretory condition in a patient undergoing treatment with a calcium channel blocker.

13. Use of a calcium channel blocker for the manufacture of a medicament for use in the treatment of a muscle tone disorder or a proliferative, inflammatory or secretory condition in a patient undergoing treatment with an anti-muscarinic agent.

14. Use according to claim 12 or claim 13, wherein the medicament is suitable for topical administration.

15. Use according to claim 12 or claim 13, wherein the medicament is suitable for inhaled administration.

16. Use according to any of claims 12 to 15, wherein the anti-muscarinic agent is glycopyrrolate.

17. Use according to any of claims 12 to 15, wherein the anti-muscarinic agent is tiotropium.

18. Use according to any of claims 12 to 17, wherein the calcium channel blocker is diltiazem.

19. Use product according to any of claims 12 to 17, wherein the calcium channel blocker is verapamil.

20. Use according to any of claims 12 to 17, wherein the calcium channel blocker is a dihydropyridine drug.

21. Use according to any of claims 12 to 20, wherein the condition is respiratory.

22. Use according to any of claims 12 to 21, for the treatment of a proliferative, inflammatory or secretory condition.
